(19) United States
(12) Patent Application Publication
(10) Pub. No.: US 2020/0178764 A1
(43) Pub. Date: Jun. 11, 2020

(54) VISUALIZATION AND SPACEMAKING DEVICES

(71) Applicant: AtriCure, Inc., Mason, OH (US)

(72) Inventors: Tamer Ibrahim, Danville, CA (US); Dwight P. Morejohn, Davis, CA (US); Matthew Monti, Cincinnati, OH (US); Kenneth L. Miller, Hamilton, OH (US); Michael J. Banchieri, Discovery Bay, CA (US); Brian Flores, Castro Valley, CA (US); Ara Stephanian, Davis, CA (US)

(73) Assignee: AtriCure, Inc., Mason, OH (US)

(21) Appl. No.: 16/709,289

(22) Filed: Dec. 10, 2019

Related U.S. Application Data
(60) Provisional application No. 62/777,836, filed on Dec. 11, 2018.

(51) Int. Cl. A61B 1/00; A61M 29/02; A61B 1/313; A61B 90/00; A61M 25/10; A61B 18/00

(52) U.S. Cl. CPC A61B 1/00082; A61M 29/02; A61B 1/00154; A61B 1/3137; A61B 2018/00577; A61B 2090/3925; A61M 25/1011; A61B 2090/3966; A61B 2090/378; A61B 2090/376

(57) ABSTRACT

Medical devices are disclosed. An example surgical spacemaking device may include an elongate element configured for insertion into a patient's body and including a distal end portion and an internal lumen, an inner element disposed generally coaxially within the internal lumen of the elongate element, an expandable element disposed generally circumferentially about a distal end portion of the inner element, and an actuator operable to reposition the expandable element between a contracted configuration and an expanded configuration. In the contracted configuration, the expandable element may have a relatively small diameter and, in the expanded configuration, the expandable element may have a relatively large diameter.

### CROSS REFERENCE TO RELATED APPLICATIONS

This application claims the benefit of U.S. Provisional Application No. 62/777,836, filed Dec. 11, 2018, which is incorporated by reference.

### INTRODUCTION TO THE INVENTION

The present disclosure is directed to medical instruments and devices and related methods, and, more specifically, to medical and surgical systems and devices for improving visualization and working space, such as in the context of minimally invasive procedures, and related methods.

The present disclosure contemplates that some surgical procedures, such as minimally invasive surgical procedures, may utilize surgical tools that are extended into a patient's body to an operative area via one or more relatively small incisions or percutaneous access points. In some such surgeries, which may include interventional cardiology and/or electrophysiology procedures, the patient's internal anatomic structures and/or the surgical tools within the patient's body may be observed indirectly (e.g., not directly through the incision) by the surgeon. For example, some minimally invasive surgical procedures may involve observing the patient's internal anatomic structures and/or the surgical tools using radiographic imaging techniques, such as fluoroscopy. Some minimally invasive surgical procedures may involve visually observing the patient's internal anatomic structures and/or the surgical tools via a camera placed within the patient's body, such as by viewing images obtained by an endoscope on an external video monitor. As used herein, “scope” may refer to an optical device used to observe an area within a patient's body and may include rigid or flexible endoscopes, laparoscopes, arthroscopes, bronchoscopes, ureteroscopes, etc. A scope may include a lighting feature and/or may be utilized with a separate lighting device, either of which may be used to illuminate the field of view of the scope. Further, scopes as described herein may include “chip on the tip” configurations in which a video sensor and/or a light source are permanently embedded into a device, such as proximate the distal end of an instrument.

The present disclosure contemplates that, in some circumstances, it may be difficult to move an endoscope into a desired position and/or to view a particular anatomic structure using an endoscope. For example, some endoscopes may be difficult to utilize in tight tissue planes.

While known devices have been used safely and effectively, improvements relating to visualization and spacemaking may be beneficial for users (e.g., clinicians) and patients.

It is a first aspect of the present disclosure to provide a surgical spacemaking device including an elongate element configured for insertion into a patient's body, the elongate element comprising a distal end portion and an internal lumen; an inner element disposed generally coaxially within the internal lumen of the elongate element, the inner element comprising a distal end portion; an expandable element disposed generally circumferentially about the distal end portion of the inner element; and/or a first actuator operable to reposition the expandable element between a contracted configuration and an expanded configuration. In the contracted configuration, the expandable element may have a relatively small diameter. In the expanded configuration, the expandable element may be expanded generally radially outward from the distal end portion of the inner element to a relatively large diameter.

In a more detailed embodiment of the first aspect, the expandable element may include a mesh tube configured to radially expand when the mesh tube is shortened and to radially contract when the mesh tube is lengthened. The mesh tube may be constructed from strands of at least one of metal and polymer. The strands may be at least one of woven and braided to form the mesh tube. The expandable element may include a flexible membrane disposed on the mesh tube. The mesh tube may be substantially closed. The mesh tube may be substantially open. The mesh tube is generally shaped as a right circular cylinder.

In a more detailed embodiment of the first aspect, in at least one of the contracted configuration and the expanded configuration, the expandable element may be arranged substantially coaxially with the distal end portion of the inner element. The expandable element may be configured to expanded at least one of uniformly circumferentially and uniformly radially away from the inner element.

In a more detailed embodiment of the first aspect, the first actuator may include a push element configured to exert a distal pushing force on a proximal portion of the expandable element and/or a pull element configured to exert a proximal pulling force on a distal portion of the expandable element. The push element may include a push tube and/or the pull element may include a suture extending through the push tube. The device may include a second actuator. The second actuator may include a respective push element configured to exert a respective distal pushing force on the proximal portion of the expandable element and/or a respective pull element configured to exert a respective proximal pulling force on the distal portion of the expandable element.

In a more detailed embodiment of the first aspect, the device may include at least one connection element extending generally radially between the distal end portion of the inner element and the expandable element. The inner element may include a scope.

It is a second aspect of the present disclosure to provide a method of forming a working space within a patient's body, the method comprising inserting a surgical device into a patient's body, the surgical device including an elongate element, the elongate element including a distal end portion, and an expandable element disposed proximate the distal end portion; and expanding the expandable element from a contracted configuration to an expanded configuration to form a working space proximate the distal end portion of the elongate element between anatomical structures within the patient's body. In the contracted configuration, the expandable element may have a relatively small diameter. In the expanded configuration, the expandable element may have a relatively large diameter.

In a more detailed embodiment of the second aspect, expanding the expandable element may include operating an actuator operable to reposition the expandable element between the contracted configuration and the expanded configuration. The expandable element may include a mesh tube configured to radially expand when the mesh tube is shortened and to radially contract when the mesh tube is lengthened. Operating the actuator may include at least one of pushing distally on a proximal end portion of the expandable element and pulling proximally on a distal end portion of the expandable element.

In a more detailed embodiment of the second aspect, expanding the expandable element may include expanding the expandable element at least one of uniformly circumferentially and uniformly radially outward. Inserting the surgical device into the patient's body may include inserting the surgical device into a pericardial space. Expanding the expandable element may include expanding the expandable element to create a working space between a parietal pericardial layer and a visceral pericardial layer. The method may include, after expanding the expandable element, occluding a left atrial appendage. The method may include, after expanding the expandable element, ablating tissue.

In a more detailed embodiment of the second aspect, the expandable element may include an inflatable element. Expanding the expandable element may include inflating the inflatable element.

It is a third aspect of the present disclosure to provide a surgical device including a scope and a first expandable element disposed proximate a distal end portion of the scope. The expandable element may be repositionable between a contracted configuration and an expanded configuration. In the expanded configuration, the expandable element may be operative to create a working space proximate the distal end portion of the scope.

In a more detailed embodiment of the third aspect, the device may include a second expandable element disposed proximally to the first expandable element. The device may include a third expandable element disposed proximally to the second expandable element.

In a more detailed embodiment of the third aspect, the first expandable element may include an inflatable element. In the expanded configuration, the inflatable element may extend distally beyond the scope. The scope may be arranged to view a target tissue through at least a portion of the inflatable element. In the expanded configuration, the inflatable element may be shaped generally in the form of a torus.

In a more detailed embodiment of the third aspect, the scope may be disposed within an elongate element comprising a sheath. The expandable element may be disposed on the sheath. The expandable element may include a mesh tube.

### BRIEF DESCRIPTION OF THE DRAWINGS

Example embodiments are described in conjunction with the accompanying drawing figures in which:

FIG. 1 is a cross-sectional view of an example surgical device in a contracted configuration;

FIG. 2 is a cross-sectional view of the surgical device of FIG. 1 in an expanded configuration;

FIG. 3 is a distal perspective view of the surgical device of FIG. 1 in an expanded configuration;

FIG. 4 is a cross-sectional view of the surgical device of FIG. 1 in an expanded configuration in a pericardial space;

FIG. 5 is a side elevation view of an alternative example surgical device in a contracted configuration;

FIG. 6 is a side elevation view of the surgical device of FIG. 5 in an expanded configuration;

FIG. 7 is a side elevation view of an alternative example surgical device;

FIG. 8 is a side elevation view of an alternative example surgical device;

FIG. 9 is a distal end perspective view of the surgical device of FIG. 8;

FIG. 10 is a distal perspective view of an alternative example surgical device;

FIG. 11 is a side perspective view of an alternative example surgical device;

FIG. 12 is a distal perspective view of the surgical device of FIG. 11;

FIG. 13 is a side elevation view of an alternative example surgical device;

FIG. 14 is a side elevation view of an alternative example surgical device; all in accordance with at least some aspects of the present disclosure.

### DETAILED DESCRIPTION

Example embodiments according to the present disclosure are described and illustrated below to encompass devices, methods, and techniques relating to medical procedures. Of course, it will be apparent to those of ordinary skill in the art that the embodiments discussed below are examples and may be reconfigured without departing from the scope and spirit of the present disclosure. It is also to be understood that variations of the example embodiments contemplated by one of ordinary skill in the art shall concurrently comprise part of the instant disclosure. However, for clarity and precision, the example embodiments as discussed below may include optional steps, methods, and features that one of ordinary skill should recognize as not being a requisite to fall within the scope of the present disclosure.

The present disclosure includes, inter alia, medical instruments and devices and related methods, and, more specifically, medical and surgical systems and devices for improving visualization and working space, such as in the context of minimally invasive procedures, and related methods. Various aspects of the present disclosure may be broadly applicable, such as to various interventional procedures including percutaneous procedures, catheter-based procedures, minimally invasive surgical procedures, and/or open surgical procedures, and/or combinations thereof. For purposes of clarity, as used herein, “surgery” or “surgical” may refer generally to any such interventional procedure, regardless of whether the procedure is performed by a surgeon, interventional radiologist, interventional cardiologist, electrophysiologist, or other clinician, and these terms should not be considered as limiting the scope or applicability of this disclosure. Some example embodiments according to at least some aspects of the present disclosure may be particularly useful in connection with cardiac procedures, such as occlusion of the left atrial appendage.

The present disclosure contemplates that some endoscopes, such as small-diameter, flexible endoscopes, may have difficulty seeing and/or moving in tight tissue planes. For example, some such endoscopes may have difficulty seeing and/or moving in the pericardial space (e.g., between the parietal and visceral pericardial layers). The present disclosure contemplates that the difficulties may result from one or more of the following: (1) when moist tissue contacts the endoscope tip, visualization may be impaired; (2) when a flexible endoscope attempts to articulate anywhere except within the tissue plane (such as to look up or down in a horizontal tissue plane) it is often not strong enough to lift the tissue out of the way to make its curved articulation; and/or (3) in the close confines of a tight space (e.g., inside the pericardium and against heart tissue), the target landmarks may not be in view before they are contacted and/or are too close for easy identification and/or orientation. Various example embodiments according to at least some aspects of the present disclosure may overcome some of these difficulties. For example, various example embodiments according to at least some aspects of the present disclosure may improve visualization near moist tissue, which may impair viewing through a scope. Also, various example embodiments according to at least some aspects of the present disclosure may be used to separate closely spaced tissues to improve visualization, such as when locating landmark target tissues for identification and orientation. Various example embodiments according to at least some aspects of the present disclosure may be used to form working spaces that may facilitate manipulation of medical devices, such as minimally invasive surgical instruments or catheter-based instruments, such as during left atrial appendage occlusion procedures.

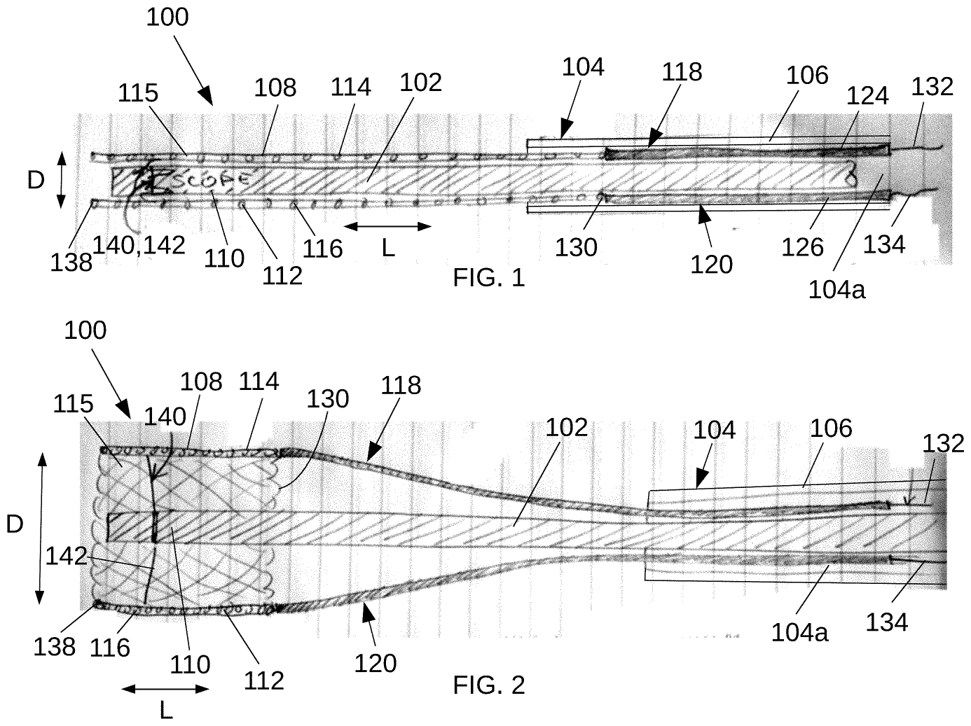

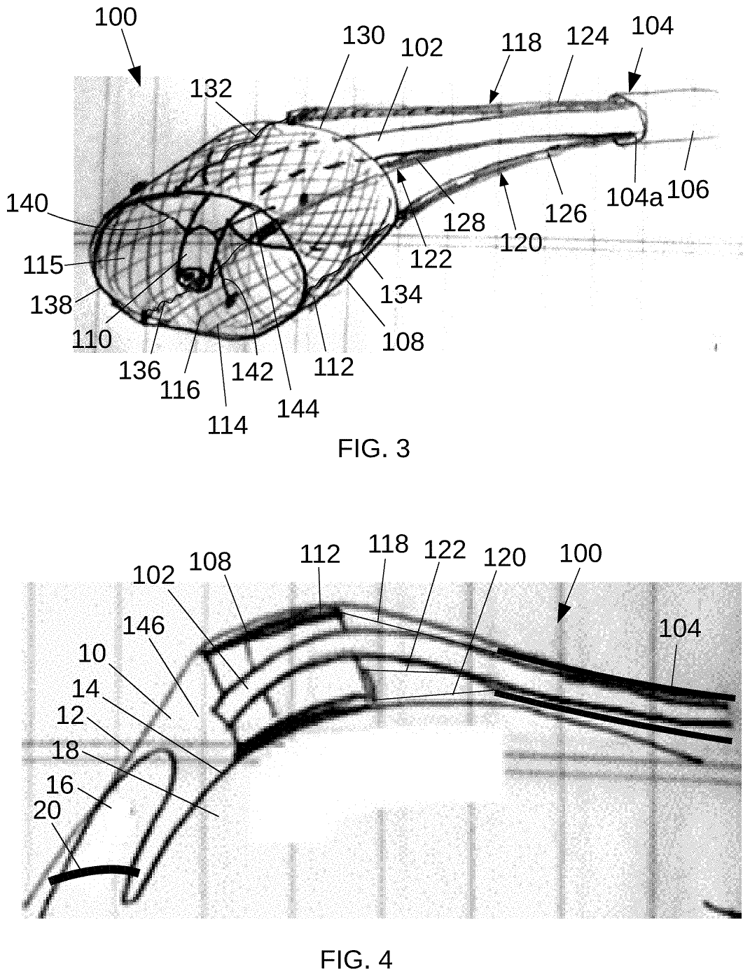

FIG. 1 is a cross-sectional view of an example surgical device 100 in a contracted configuration, FIG. 2 is a cross-sectional view of the surgical device 100 in an expanded configuration, and FIG. 3 is a distal perspective view of the surgical device 100 in an expanded configuration, all according to at least some aspects of the present disclosure. As used herein, “distal” may refer generally to the direction towards the portion of a surgical device that is inserted into a patient's body, and “proximal” may refer generally to the direction towards the portion of a surgical device that remains outside of the patient's body.

Referring to FIGS. 1-3, the example surgical device 100 may include an inner element 102 disposed generally coaxially within an elongate element 104. The elongate element 104 may be generally cylindrical. The elongate element 104 may include a proximal portion 106, which may be flexible or rigid and/or which may have a generally smooth outer surface which may facilitate insertion into and/or movement within a patient's body.

In some example embodiments, the elongate element 104 may be generally tubular (e.g., including an internal lumen 104a), such as to receive the inner element 102 therein. For example, an elongate element 104 including an internal lumen 104a may include a tubular sheath and/or a scope including a working channel. The internal lumen 104a may receive an inner element 102, such as a scope and/or other surgical instrument, therein.

In some example embodiments, the elongate element 104 may not include an accessible internal lumen 104a. For example, the elongate element 104 may include a scope or other surgical instrument that does not include a working channel. Accordingly, some such embodiments may not include an inner element 102.

Generally, an inner element 102 according to the present disclosure may include any desired tool or instrument that may be useful in a particular procedure. Example inner elements 102 may include, for example and among other things, components including one or more sensors (e.g., temperature, conductivity, impedance, pressure, etc.), one or more surgical tools (e.g., graspers, scissors, clamps, staplers, etc.), one or more ablation devices (e.g., pens, clamps, etc.), and/or one or more implantable elements (e.g., clips, clamps, lariats, staples, sutures, etc.). U.S. Pat. No. 8,034,051, which is incorporated by reference herein, discloses various example devices and methods that may be utilized in connection with embodiments according to at least some aspects of the present disclosure.

The elongate element 104 may include a distal end portion 108, which may be disposed distally from the proximal portion 106. The distal end portion 108 of the elongate element 104 may be disposed generally circumferentially about a distal end portion 110 of the inner element 102. The surgical device 100 may include an expandable element 112, which may be configured to transition between a contracted configuration (e.g., FIG. 1) and an expanded configuration (e.g., FIGS. 2 and 3).

In the example embodiment illustrated in FIGS. 1-3, the example expandable element 112 may be disposed at the distal end portion 108 of the elongate element 104. The expandable element 112 may be generally tubular and/or generally in the shape of a right circular cylinder. The expandable element 112 may be configured to expand generally radially outward, such as radially outward from the inner element 102. For example, as shown in FIG. 1, the expandable element 112 may have a relatively small diameter (e.g., a maximum dimension in a diametrical direction D) when in the contracted configuration. As shown in FIG. 2, the expandable element 112 may have a relatively large diameter when in the expanded configuration. The expandable element 112 may be generally arranged coaxially with the inner element 102 in the contracted configuration and/or the expanded configuration. The expandable element 112 may be configured to expand generally uniformly circumferentially and/or uniformly radially, such as radially away from the inner element 102.

In the example embodiment illustrated in FIGS. 1-3, the example expandable element 112 may include an elastically deformable material formed generally into a mesh tube 114. For example, the mesh tube 114 may be constructed of strands 116 of metal (e.g., a nickel titanium alloy) and/or polymer that may be woven and/or braided. The mesh tube 114 may be formed so that some of the strands 116 are generally helically wound and/or at least some of the strands are arranged obliquely (e.g., non-parallel and non-perpendicular) when the expandable element 112 is in at least some configurations. The mesh tube 114 may be closed, such as with a flexible membrane 115 (e.g., a thin layer of silicone) disposed on and/or covering the spaces between the wires or strands 116. In some embodiments, the mesh may be open (e.g., the spaces between the wires or strands may be generally unobstructed).

In the example embodiment illustrated in FIGS. 1-3, the mesh tube 114 may be configured to radially expand and/or contract as the longitudinal length (e.g., in the longitudinal direction L) is changed. For example, the mesh tube 114 may be configured to operate generally in the manner of a Chinese finger trap. Lengthening the mesh tube 114 may cause its diameter to narrow and/or shortening the mesh tube 114 may cause its diameter to expand. In some example embodiments, the mesh tube 114 may be constructed so that it is biased toward either the expanded or the contracted configuration (e.g., without externally applied forces, the mesh tube 114 may tend to self-expand and/or self-contract to a particular configuration).

In the example embodiment illustrated in FIGS. 1-3, the mesh tube 114 of the expandable element 112 may be shortened and/or lengthened (e.g., expanded and/or contracted) using one or more actuators 118, 120, 122. Each actuator 118, 120, 122 may include a respective push element 124, 126, 128, each of which may be arranged to exert a distal, pushing force on a proximal portion 130 of the mesh tube 114. Each actuator 118, 120, 122 may include a respective pull element 132, 134, 136, each of which may be arranged to exert a proximal, pulling force on a distal portion 138 of the mesh tube 114. For example, the pull elements 132, 134, 136 may be constructed from sutures (or other similar materials). The push elements 124, 126, 128 may be constructed from relatively small diameter tubes, through which the pull elements 132, 134, 136 may extend.

Distal movement of the push elements 124, 126, 128 relative to the pull elements 132, 134, 136 and/or proximal movement of the pull elements 132, 134, 136 relative to the push elements 124, 126, 128 may cause the mesh tube 114 to shorten, thereby circumferentially and/or radially expanding the expandable element 112. Similarly, proximal movement of the push elements 124, 126, 128 relative to the pull elements 132, 134, 136 and/or distal movement of the pull elements 132, 134, 136 relative to the push elements 124, 126, 128 may cause the mesh tube 114 to lengthen, thereby circumferentially and/or radially contracting the expandable element 112.

Generally, the proximal and/or distal movement of the push elements 124, 126, 128 and/or the pull elements 132, 134, 136 may be effected by operatively coupling the push elements 124, 126, 128 and/or the pull elements 132, 134, 136 to one or more operator-controlled input devices. For example, a lever, trigger, switch, button, plunger, or similar component may be used to operate the actuators 118, 120, 122 to expand and/or contract the expandable element 112.

In some example embodiments, the distal end portion 110 of the inner element 102 may be coupled to the expandable element 112 by one or more connection elements 140, 142, 144. For example, the connection elements 140, 142, 144, which may comprise sutures, may suspend the distal end portion 110 of the inner element 102 generally centered (e.g., coaxially) within the expandable element 112, such as by extending between the inner element and the radially inner or outer aspect of the expandable element 112.

In some example embodiments, the inner element 102 may include a scope, which may be steerable by the operator from a proximal portion outside of the patient's body. In some example embodiments, the inner element 102 may include an access sheath through which surgical tools may be inserted.

Generally, it is within the scope of the disclosure to utilize alternative expandable elements disposed at different longitudinal locations on a surgical device, expandable elements having different shapes (e.g., generally frustoconical, generally spherical, etc.), and/or expandable elements that are configured to expand non-coaxially and/or non-uniformly (e.g., more to one side than another).

FIG. 4 is a cross-sectional view of the example surgical device 100 in a pericardial space 10 (e.g., between the parietal 12 and visceral 14 pericardial layers), according to at least some aspects of the present disclosure. In an example surgical procedure, the target tissue may include the left atrial appendage 16 of the patient's heart 18. For example, the surgical device 100 may be used in connection with placement of a left atrial appendage occlusion device 20 on the left atrial appendage 16.

The surgical device 100 may be positioned in the patient's body so that the distal end portion 108 of the elongate element 104 is at a desired location. For example, the distal end portion 108 of the elongate element 104 may be positioned within the pericardial space 10 and/or oriented generally facing toward the left atrial appendage 16. In some example embodiments, the surgical device 100 may be positioned within a patient using a medical imaging technique, such as fluoroscopy and/or ultrasound. Accordingly, the surgical device 100 may be constructed so that one or more components thereof is readily visible using the selected imaging technique, such as by utilizing radiopaque materials and/or echogenic markers.

The expandable element 112 may be expanded, such as by operation of actuators 118, 120, 122. Such expansion may separate nearby tissues (e.g., the parietal 12 and visceral 14 pericardial layers) to form a working space 146, which may facilitate visualization of anatomical structures. For example, in an embodiment including a deflectable scope as an inner element 102, the scope may be able to rotate and deflect within the space formed by the expanded expandable element 112, with limited or no movement of the elongate element 104. In the illustrated example, a scope comprising inner element 102 may visualize the left atrial appendage 16. The left atrial appendage occlusion device 20 may be delivered and/or placed using the surgical device 100. Alternatively, the surgical device 100 may be used to facilitate visualization and/or formation of a working space 146, allowing the left atrial appendage occlusion device 20 to be delivered and/or placed using another surgical tool.

In some example procedures, the expandable element 112 may be expanded and/or contracted 112 more than once, such as to facilitate repositioning of the surgical device 100 and/or formation of working spaces 146 at various locations within the patient's body. In some example procedures, the expandable element 112 may be contracted after the target tissue has been reached. For example, in an example procedure involving the left atrial appendage 16, the expandable element 112 may be contracted after the left atrial appendage 16 has been identified and/or engaged by a grasper, but before the left atrial appendage occlusion device 20 is placed on the left atrial appendage 16.

Described below are various alternative example surgical devices. Unless specifically indicated, the description of the structure and function or methodology of corresponding components with respect to the surgical device 100 may apply to the following alternative embodiments, and the description of the structure and function or methodology of corresponding components with respect to the following alternative embodiments may apply to the surgical device 100 and/or to the other alternative embodiments. Generally, corresponding reference numerals refer to similar structures. Therefore, repeated explanation of structure and function or methodology described elsewhere herein is not necessary and may be omitted for brevity.

Some alternative example surgical devices may include expandable elements including inflatable elements. Generally, in various example embodiments, expandable elements such as inflatable elements may facilitate visualization, spacemaking, positioning, and/or holding a component in a desired position, for example. Expandable elements such as inflatable elements may be disposed at various locations (e.g., axially and/or circumferentially) on various example surgical devices.

Some example inflatable elements may be constructed of materials having elastomeric properties that allow an inflatable element to stretch and/or return to substantially its original size and/or shape. Some example inflatable elements may be constructed of substantially inelastic materials so that an inflatable element may inflate and deflate substantially without stretching. Inflatable elements may be configured to inflate to any desired shape, such as generally rounded, generally spherical, and/or generally toroidal shapes as described below.

Some example inflatable elements may be constructed from materials that may be at least partially opaque. Some example inflatable elements may be constructed from materials that may be at least partially transparent, such as substantially optically clear. For example, inflatable elements through which a scope may visualize an anatomical structure may be constructed of optically clear plastic. Inflatable elements that are not configured to interpose a visualization device and an anatomical structure may be constructed from materials that may not be optically clear.

In various example embodiments, an inflatable element may be expanded and/or contracted (e.g., inflated and/or deflated) by delivering fluid(s) to and/or withdrawing fluid(s) from an interior volume of the inflatable element. As used herein, fluid may refer to any liquid or gas (or mixture thereof) that may be acceptable for use in a surgical environment, such as water, saline solution, air, nitrogen, carbon dioxide, etc. In some example embodiments, a fluid used to expand an inflatable structure may include a contrast agent to improve visibility using a medical imaging technique (e.g., fluoroscopy and/or ultrasound).

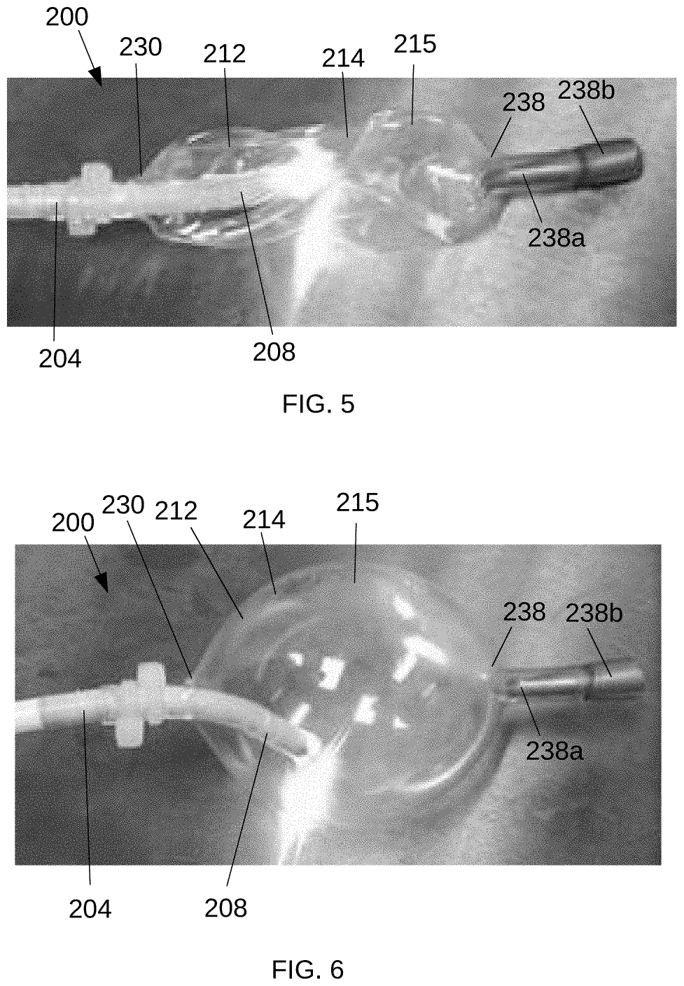

FIG. 5 is a side elevation view of an alternative example surgical device 200 in a contracted configuration and FIG. 6 is a side elevation view of the surgical device 200 in an expanded configuration, according to at least some aspects of the present disclosure. Generally, in the example surgical device 200, the expandable element 212 may include an inflatable element 214. The inflatable element 214 may include a generally spherically shaped, flexible membrane 215 that expands outward at the distal end portion 208 of an elongate element 204, such as a flexible scope. For example, a proximal end portion 230 of the inflatable element 214 may be sealed to the flexible scope. The distal end portion 238 of the inflatable element 214 may extend distally beyond the flexible scope and may be substantially continuous with the other portions of the inflatable element 214 (e.g., like the rounded end of a balloon). Alternatively, as illustrated in FIGS. 5 and 6, the distal end portion 238 of the inflatable element 214 may include an opening 238a sealed by a sealing element 238b (e.g., a plug).

In some example embodiments, when the expandable element 212 is in the expanded configuration (FIG. 6), for example when the inflatable element 214 is inflated, the distal end portion 208 of the elongate element 204 may be deflected generally within the expandable element 212. This may facilitate visualization of the anatomical structures at various positions around the expandable element 212. For example, a flexible scope may be used to view anatomical structures through an at least partially transparent inflatable element 214.

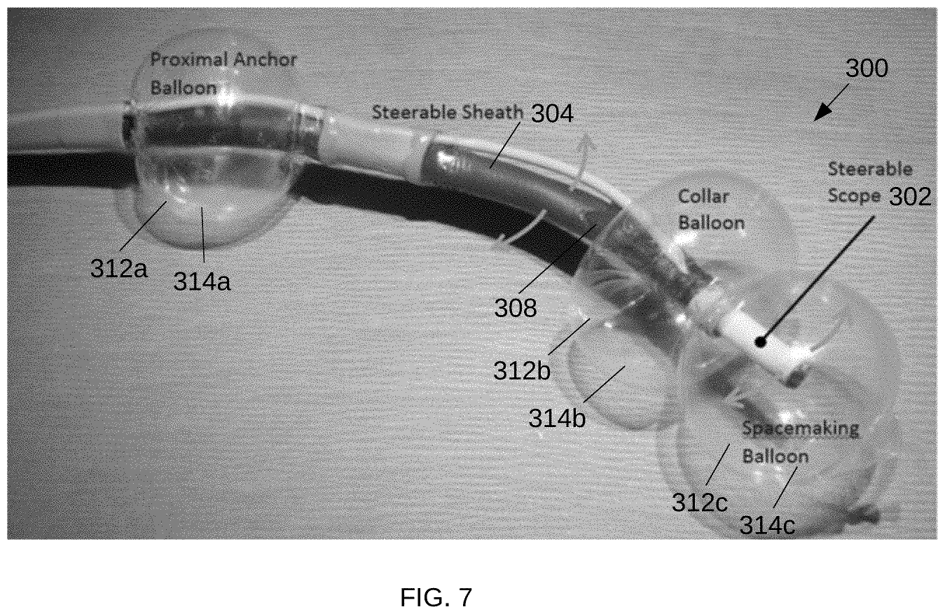

FIG. 7 is a side elevation view of an alternative example surgical device 300, according to at least some aspects of the present disclosure. Generally, in this example embodiment, one or more expandable elements 312a, 312b, 312c may be disposed on an elongate element 304 (e.g., a steerable sheath). The steerable sheath may be steered to selectively orient the opening plane of the expandable elements 312a, 312b, 312c, particularly the distal-most expandable element 312c, using push/pull steering elements within the sheath (e.g., pull on one side and/or push on the other). An inner element 302 (e.g., a steerable scope) may extend through the internal lumen of the elongate element 304. In some example embodiments, the expandable elements 312a, 312b, 312c may be individually expandable and/or contractable and/or the expandable elements 312a, 312b, 312c may be operated together between the expanded and/or contracted configurations.

The surgical device 300 may include a proximal anchor expandable element 312a, which may be used to aid in positioning the elongate element 304 and/or securing the elongate element 304 in a desired position. The proximal anchor expandable element 312a may include a proximal anchor inflatable element 314a, which may be generally similar to other inflatable elements described herein except that it may be positioned along the sheath 304 at some distance proximal to the distal end portion 308 of the elongate element 304. In this example embodiment, the proximal anchor expandable element 312a may be configured to expand generally uniformly radially outwardly from the elongate element 304 and/or may form a generally spherical shape in the expanded configuration.

The surgical device 300 may include a collar expandable element 312b, which may be used to aid in positioning the surgical device 300, such as in the pericardial space 10 (FIG. 4). The collar expandable element 312b may include a collar inflatable element 314b, which may be generally similar to other inflatable elements described herein except that it may be positioned along the elongate element 304 proximate the distal end portion 308 of the elongate element 304. In this example embodiment, the collar expandable element 312b may be configured to expand generally uniformly radially outwardly from the elongate element 304 and/or may form a generally spherical shape in the expanded configuration.

The surgical device 300 may include a spacemaking expandable element 312c, which may be used to facilitate visualization and/or to form a working space, such as a working space 146 within the pericardial space 10 (FIG. 4). The spacemaking expandable element 312c may include a spacemaking inflatable element 314c, which may be generally similar to other inflatable elements described herein except that it may be positioned distally on the elongate element 304, such as extending distally beyond the distal end portion 308 of the elongate element 304. In this example embodiment, the spacemaking expandable element 312c may be configured to expand generally uniformly radially outwardly from the elongate element 304 and/or may form a generally spherical shape in the expanded configuration. The inner element 302 (e.g., a steerable scope) may extend distally beyond the internal lumen of the elongate element 304 and/or into the internal volume of the spacemaking expandable element 312c, which may allow deflection of the scope generally within the spacemaking expandable element 312c. This may facilitate visualization of the anatomical structures at various positions around the spacemaking expandable element 312c. For example, a scope may be used to view anatomical structures through an at least partially transparent spacemaking inflatable element 314c.

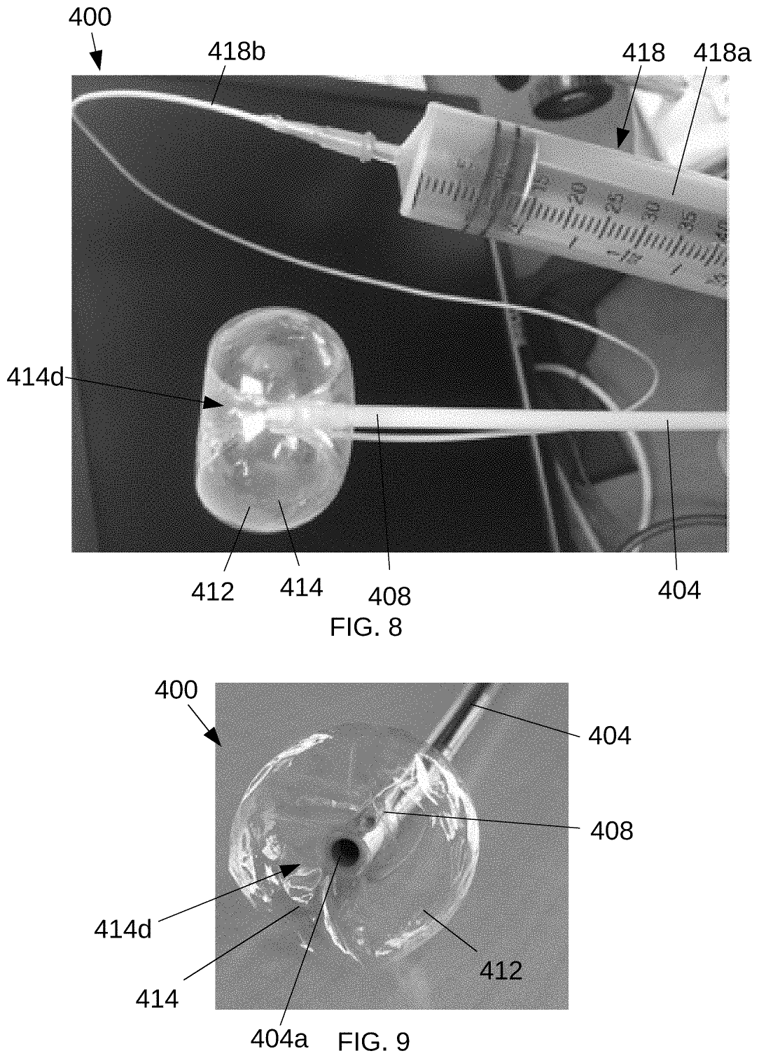

FIG. 8 is a side elevation view of an alternative example surgical device 400 and FIG. 9 is a distal end perspective view the surgical device 400, according to at least some aspects of the present disclosure. Generally, in this example embodiment, an expandable element 412 may be disposed proximate a distal end portion 408 of an elongate element 404. The expandable element 412 may include an inflatable element 414 generally in the shape of a torus, which may be disposed generally circumferentially about the distal end portion 408 of the elongate element 404. Because the central opening 414d of the expandable element 412 is generally coaxially aligned with the internal lumen 404a of the elongate element 404, a surgical tool extending through the internal lumen 404a of the elongate element 404 may not be obstructed in the distal direction by the expandable element 412. Accordingly, this example embodiment may be used in connection with one or more inner elements (e.g., scopes and/or other surgical instruments) that may require direct access to anatomical structures (e.g., target tissues) within the patient's body, such as tissue manipulation instruments.

FIG. 8 also illustrates an alternative example actuator 418, which may be used in connection with any inflatable elements disclosed herein. The actuator 418 may include a syringe 418a fluidically coupled to the interior volume of the inflatable element 414. Movement of the syringe's plunger may be operative to expand or contract the expandable element 412 by delivering fluid to and/or withdrawing fluid from the interior volume of the inflatable element, such as via a supply tube 418b.

Generally, an example method of using an example surgical device including an expandable element including an inflatable element may include approaching a target tissue with the surgical device. The inflatable element may be inflated in the tissue plane. Once inflated, the inflatable element may rotate omnidirectionally. The inflated inflatable element may require only a small amount of force to rotate because the inflatable element may be generally smooth and/or may be lubricated from available fluid. The surgical device may facilitate scope rotation and viewing changes in rotation and roll. The scope may change its direction of view with much freedom and distance to the tissues because it may not be directly in tissue contact. Optionally, a space may be available in front of the scope for visualization.

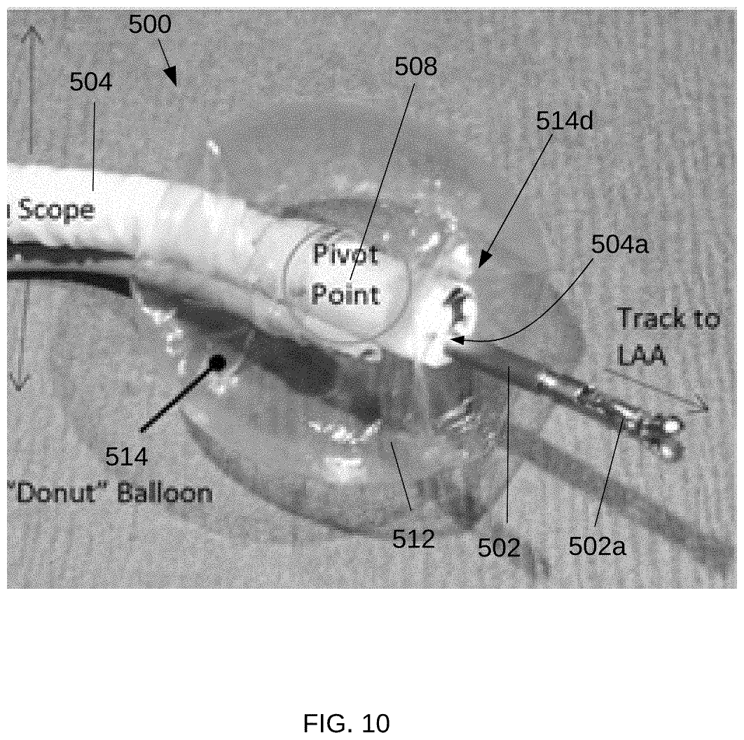

FIG. 10 is a distal perspective view of an alternative example surgical device 500, according to at least some aspects of the present disclosure. Generally, the surgical device 500 may be similar to the surgical device 400 of FIGS. 8 and 9, except that the elongate element 504 may include a flexible scope with an internal lumen 504a (e.g., working channel) extending longitudinally therethrough. The internal lumen 504a may be used to direct an inner element 502, such as a surgical instrument 502a for tissue manipulation (e.g., a grasper), to a target tissue (e.g., a left atrial appendage 16 (FIG. 4)). The internal lumen 504a may be used to infuse and/or aspirate a fluid.

Generally, in this example embodiment, an expandable element 512 may be disposed proximate a distal end portion 508 of the elongate element 504. The expandable element 512 may include an inflatable element 514 generally in the shape of a torus, which may be disposed generally circumferentially about the distal end portion 508 of the flexible scope. Because the central opening 514d of the expandable element 512 is generally coaxially aligned with the elongate element 504, an internal element 502 extending through the internal lumen 504a of the elongate element 504 and/or fluids moving into or out of the internal lumen 504a of the elongate element 504 may not be obstructed in the distal direction by the expandable element 512.

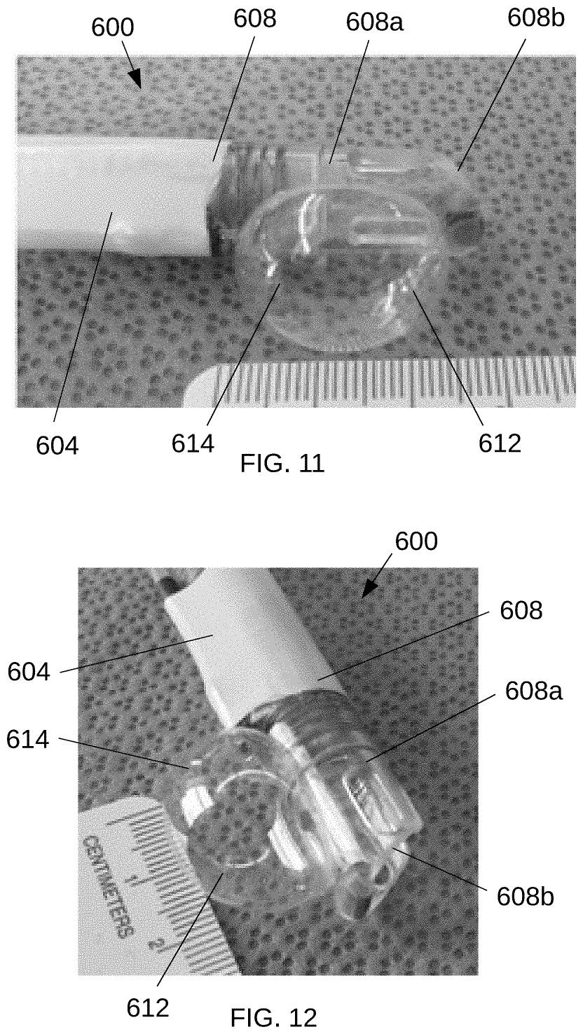

FIG. 11 is a side perspective view of an alternative example surgical device 600 and FIG. 12 is a distal perspective view of the surgical device 600, according to at least some aspects of the present disclosure. Generally, in this example embodiment, the surgical device 600 may include an expandable element 612 configured to expand generally laterally from one side of a distal end portion 608 of an elongate element 604. The distal end portion 608 of the elongate element 604 may include a tip 608a, which may be substantially transparent and/or which may include an angled distal face 608b. The expandable element 612, which may include an inflatable element 614, may be disposed generally on a lateral side of the tip 608a, such as a side facing generally away from the angled distal face 608b.

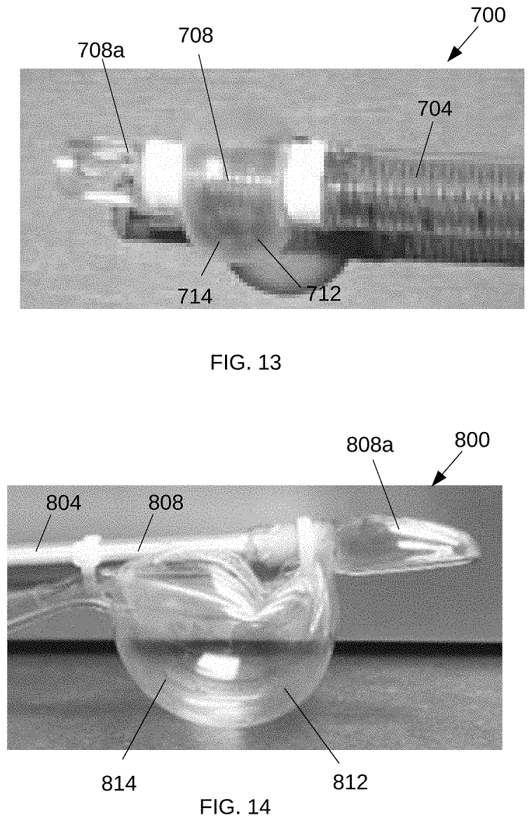

FIG. 13 is a side elevation view of an alternative example surgical device 700, according to at least some aspects of the present disclosure. Generally, in this example embodiment, the surgical device 700 may include a distal end portion 708 of a elongate element 704 including a tip 708a generally similar to the tip 608a of FIGS. 11 and 12. An expandable element 712 (e.g., an inflatable element 714) may be disposed generally circumferentially around the distal end portion 708 of the elongate element 704 generally proximal to the tip 708a.

FIG. 14 is a side elevation view of an alternative example surgical device 800, according to at least some aspects of the present disclosure. Generally, in this example embodiment, the surgical device 800 may include an expandable element 812 (e.g., an inflatable element 814) disposed on a lateral side of a distal end portion 808 of an elongate element 804 proximal to a hood 808a (e.g., a scope hood).

Some example embodiments according to the present disclosure may include a plurality of internal lumens. For example, separate lumens may be provided for different instruments and/or to facilitate infusion and/or suction of fluids.

Some example embodiments may include expandable elements utilizing combinations of different expandable elements disclosed herein. For example, such an expandable element may include one or more mesh tubes and/or one or more inflatable elements. In some such example embodiments, a push/pull type actuator assembly may not be included.

Following from the above description and invention summaries, it should be apparent to those of ordinary skill in the art that, while the methods and apparatuses herein described constitute example embodiments according to the present disclosure, it is to be understood that the scope of the disclosure contained herein is not limited to the above precise embodiments and that changes may be made without departing from the scope as defined by the following claims. Likewise, it is to be understood that it is not necessary to meet any or all of the identified advantages or objects disclosed herein in order to fall within the scope of the claims, since inherent and/or unforeseen advantages may exist even though they may not have been explicitly discussed herein.

What is claimed is:

1. A surgical spacemaking device, comprising:
an elongate element configured for insertion into a patient's body, the elongate element comprising a distal end portion and an internal lumen;
an inner element disposed generally coaxially within the internal lumen of the elongate element, the inner element comprising a distal end portion;
an expandable element disposed generally circumferentially about the distal end portion of the inner element; and
a first actuator operable to reposition the expandable element between a contracted configuration and an expanded configuration;
wherein, in the contracted configuration, the expandable element has a relatively small diameter; and
wherein, in the expanded configuration, the expandable element is expanded generally radially outward from the distal end portion of the inner element to a relatively large diameter.

2. The device of claim 1, wherein the expandable element comprises a mesh tube configured to radially expand when the mesh tube is shortened and to radially contract when the mesh tube is lengthened.

3. The device of claim 2, wherein the mesh tube is constructed from strands of at least one of metal and polymer.

4. The device of claim 3, wherein the strands are at least one of woven and braided to form the mesh tube.

5. The device of claim 2 wherein the expandable element further comprises a flexible membrane disposed on the mesh tube.

6. The device of claim 5, wherein the mesh tube is substantially closed.

7. The device of claim 2, wherein the mesh tube is substantially open.

8. The device of claim 2, wherein the mesh tube is generally shaped as a right circular cylinder.

9. The device of claim 1, wherein, in at least one of the contracted configuration and the expanded configuration, the expandable element is arranged substantially coaxially with the distal end portion of the inner element.

10. The device of claim 1, wherein the expandable element is configured to expanded at least one of uniformly circumferentially and uniformly radially away from the inner element.

11. The device of claim 1, wherein the first actuator comprises
a push element configured to exert a distal pushing force on a proximal portion of the expandable element; and
a pull element configured to exert a proximal pulling force on a distal portion of the expandable element.

12. The device of claim 11,
wherein the push element comprises a push tube; and
wherein the pull element comprises a suture extending through the push tube.

13. The device of claim 12,
further comprising a second actuator;
wherein the second actuator comprises a respective push element configured to exert a respective distal pushing force on the proximal portion of the expandable element; and
wherein the second actuator comprises a respective pull element configured to exert a respective proximal pulling force on the distal portion of the expandable element.

14. The device of claim 1, further comprising at least one connection element extending generally radially between the distal end portion of the inner element and the expandable element.

15. The device of claim 1, wherein the inner element comprises a scope.

16. A method of forming a working space within a patient's body, the method comprising:
inserting a surgical device into a patient's body, the surgical device comprising an elongate element, the elongate element comprising a distal end portion, and an expandable element disposed proximate the distal end portion; and
expanding the expandable element from a contracted configuration to an expanded configuration to form a working space proximate the distal end portion of the elongate element between anatomical structures within the patient's body;
wherein, in the contracted configuration, the expandable element has a relatively small diameter; and
wherein, in the expanded configuration, the expandable element has a relatively large diameter.

17.-23. (canceled)

24. A surgical device, comprising:
a scope; and
a first expandable element disposed proximate a distal end portion of the scope;
wherein the expandable element is repositionable between a contracted configuration and an expanded configuration; and
wherein, in the expanded configuration, the expandable element is operative to create a working space proximate the distal end portion of the scope.

25. The device of claim 24, further comprising a second expandable element disposed proximally to the first expandable element.

26. The device of claim 25, further comprising a third expandable element disposed proximally to the second expandable element.

27. The device of claim 24, wherein the first expandable element comprises an inflatable element.

28.-31. (canceled)
